(19) United States
(12) Patent Application Publication
(10) Pub. No.: US 2007/0149892 A1
(43) Pub. Date: Jun. 28, 2007

(54) Apparatus for neuromuscular function signal acquisition

(75) Inventor: Eric Guldalian, Philadelphia, PA (US)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(73) Assignee: NEUROTRON MEDICAL INC.

(21) Appl. No.: 11/316,127

(22) Filed: Dec. 22, 2005

(51) Int. Cl. A61B 5/05; A61B 5/04

(52) U.S. Cl. 600/554; 600/546

(57) ABSTRACT

An apparatus for neuromuscular functional signal acquisition is provided for evaluation of neuromuscular function. A preferred embodiment of the apparatus for neuromuscular functional signal acquisition includes an active electrode, reference electrode, ground electrode, a rotating template with pre-punched holes and a mechanical use limiter. The apparatus for neuromuscular functional signal acquisition is used in conjunction with a waveform generator, such as a nerve conduction monitor or NCV/EMG device for the detection of neuromuscular responses. The apparatus is preferably used with a stimulator probe template and mechanical use limiter to facilitate inexpensive, efficient and accurate placement of a stimulation probe.

### FIELD OF THE INVENTION

The invention relates to an apparatus for neuromuscular functional signal acquisition. Namely, the invention relates to an apparatus for evaluating sensory, motor and F-wave response of various nerves.

### BACKGROUND OF THE INVENTION

Currently available neurosensors have many deficiencies. Neurosensors available now require measuring a fixed distance proximally from an active electrode to establish a proper location for stimulating a nerve. Measuring a fixed distance manually can make replication difficult resulting in inaccurate results. Moreover, ensuring proper measurements manually leads to inefficiency and more time to apply the electrodes properly.

In addition, re-use of the neurosensors from subject to subject are limited to ensure accurate measurements when evaluating neuromuscular function. The neurosensors include electrodes that contain an electrochemical gel that is intended for multiple applications, but not multiple subjects. Once the electrode is applied to a subject and removed to evaluate all of the involved nerves, the functionality of the electrode may be compromised by 1) the electrochemical gel being exposed to the open air for an extended or indefinite period of time; 2) increased electrical impedance from foreign particles, such as skin cells and natural oils released by a subject, adhering to the electrochemical gel; and 3) physical distortion associated with application and removal from multiple subjects. Also, limiting re-use of the neurosensor electrodes from subject to subject is advantageous for sanitary reasons.

Some neurosensors currently available try to address limiting re-use of the electrodes, but have many shortcomings. One such neurosensor, attempts to limit re-use by using a sensor's on-board memory chip. The memory chip stores multiple points of information. One of the points of information is a flag for ‘used’ and ‘not used’. However, the limiter variable remains dynamic because the electronics are capable of reading and writing data whenever instructed. For example, the limiter flag can be changed from ‘used’ to ‘not used.’ Therefore, there is no way to ensure limited use of neurosensors.

As a result, there is a need in the art for an inexpensive, more accurate alternative to currently available neurosensors for evaluating neuromuscular function. Specifically, there is a need in the art for a neurosensor that can be applied more efficiently and ensures limited re-use of the electrodes in the neurosensor.

### SUMMARY OF THE INVENTION

The present invention provides an apparatus for neuromuscular functional signal acquisition in a cheaper, more efficient and more accurate fashion than is currently available in the market. Evaluation of neuromuscular function requires the release of an electrical stimulus or impulse through an anode and cathode that initiates a chemical reaction in a nerve for the purpose of generating and recording an action potential. Each neuromuscular response is captured using a set of non-invasive recording electrodes: active, reference, and ground.

To ensure proper placement of the stimulation probes relative to the electrodes for an accurate measurement of the action potential, a template is provided. The template may be fastened to the active electrode. The template contains pre-configured holes or other visual indicators for quick and accurate placement of a stimulation probe.

Furthermore, a mechanical limiter is provided for limiting re-use of the apparatus. The mechanical device is unidirectional and cannot be physically or electronically reversed. Once the mechanical limiter indicates ‘used,’ the value lies in a permanent state and the sensor can never be used again unless the limiter mechanism is physically replaced. As such, the mechanical limiter is a more secure apparatus for ensuring limited re-use of the apparatus compared to other available technologies.

Finally, the apparatus is multi-modal and compatible with all three test modes. The neurosensor can test patients for sensory, motor and F-wave responses of various nerves. The neurosensor can be used on various nerves such as, the median, ulnar, peroneal, sural, tibial and lateral plantar nerves.

As summarized, an apparatus is provided for cheaply, more efficiently and more accurately acquiring neuromuscular functional signals than what is currently available. These and other features and objects of the invention will be more fully understood from the following detailed description, which should be read in light of the accompanying drawings.

### BRIEF DESCRIPTION OF THE FIGURES

The accompanying drawings, which are incorporated in and form a part of the specification, illustrate the embodiments of the present invention and, together with the description serve to explain the principles of the invention. In the drawings:

FIG. 1 is an illustration of a conventional method for determining a stimulation point of peripheral nerves.

FIG. 2 is an illustration depicting the apparatus of the invention in use for determining the stimulation point of peripheral nerves.

FIG. 3 is an illustration of alternate embodiments of the invention in use at other nerves.

FIG. 4 is a planar view of an embodiment of the apparatus of the invention.

FIG. 5 is a planar view depicting the templates for determining the stimulation point of an ulnar nerve across an elbow.

FIG. 6 is a planar view depicting the ambidextrous templates.

### DETAILED DESCRIPTION OF THE INVENTION

Disclosed in the present invention is an apparatus and method to evaluate neuromuscular function. The apparatus and method provided herein, illustrates in detail, a cheaper, more efficient and more accurate way to evaluate neuromuscular function than what is currently available on the market. In describing a preferred embodiment of the invention illustrated in the drawings, specific terminology will be used for the sake of clarity. However, the invention is not intended to be limited to the specific terms so selected, and it is to be understood that each specific term includes all technical equivalents which operate in a similar manner to accomplish a similar purpose.

Evaluation of neuromuscular function requires the release of an electrical stimulus or impulse through an anode and cathode that initiates a chemical reaction in a nerve for the purpose of generating and recording an action potential. Each neuromuscular response is captured using a set of non-invasive recording electrodes: active, reference, and ground.

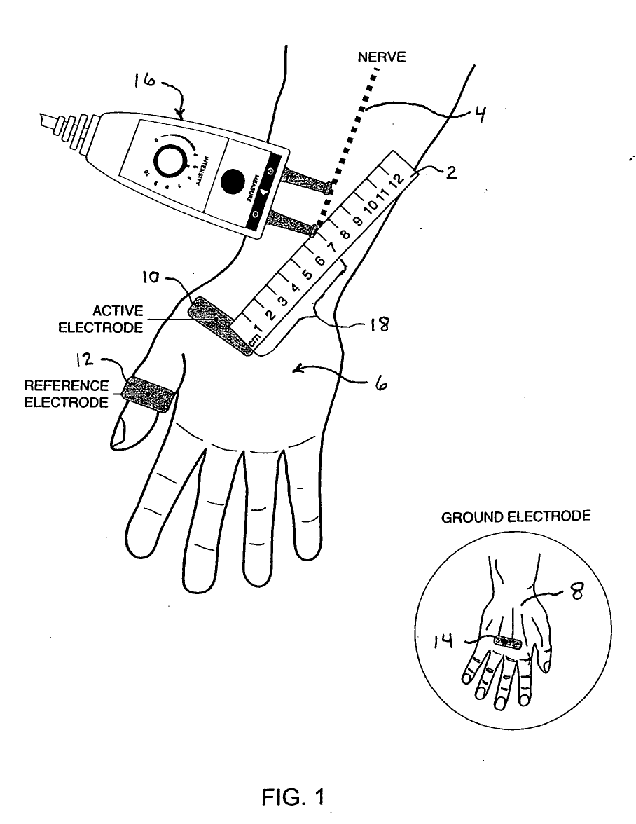

An illustration depicting a conventional method for determining a stimulation point of peripheral nerves at the wrist is shown in FIG. 1. An active electrode 10 is placed on the subject's palm 6. A reference electrode 12 is placed on the thumb. Finally, a ground electrode 14 is placed on the back of the subject's hand 8. In conventional methods, a ruler 2 is used to measure a first distance 18 from the active electrode 10. A waveform generator 16 is placed on the nerve 4 at the first distance 18 to evaluate neuromuscular function.

The conventional method of determining the stimulation point of peripheral nerves has many shortcomings. Measuring a fixed distance manually can make replication difficult resulting in inaccurate results. Moreover, ensuring proper measurements manually leads to inefficiency and more time to apply the electrodes and simulation probes properly.

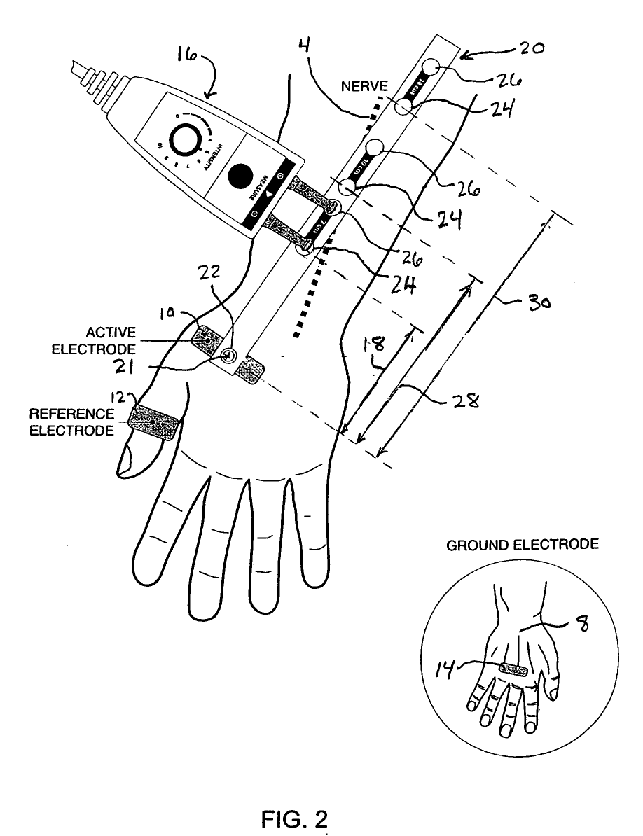

An illustration depicting an improved method for determining the stimulation point of peripheral nerves is shown in FIG. 2. An active electrode 10 is placed on the subject's palm 6. A reference electrode 12 is placed on the thumb. Finally, a ground electrode 14 is placed on the back of the subject's hand 8. Unlike the ruler 2 used in conventional methods as depicted in FIG. 1, the improved method uses a template 20. Template 20 is preferably fastened to the active electrode 10 by a fastener 22 at reference point 21. The fastener 22 allows template 20 to rotate freely for accurate placement. The template may also be fastened to the electrode in a fixed manner. A plurality of holes 24, 26 are pre-punched in template 20 for receiving the stimulation probes of waveform generator 16 constituting a guide or visual indicator for accurate placement of the probes. The waveform generator 16 can be, for example, a nerve conduction monitor or NCV/EMG device.

The visual indicators are preferably holes spaced on the template at predetermined intervals, but may be other types of indicators such as printed marks, notches or the like. Each of the plurality of pre-punched holes 24, 26 are preferably marked to receive either the simulator probe's anode or cathode. For example, the plurality of pre-punched holes 24 receives the anode of the waveform generator 16 and the plurality of pre-punched holes 26 receives the cathode of the waveform generator 16. The distance between the stimulation probe's anode and cathode on waveform generator 16 will correspond to the distance between pre-punched holes 24 and pre-punched holes 26. For example, if the distance from the center point of the stimulator's anode to the center point of the cathode is 2 cm, it will align with pre-punched holes 24 and pre-punched holes 26, respectively, that are also 2 cm on center.

Moreover on template 20 are markings indicating various distances. The distances are measured from the reference point 21. For example as illustrated in FIG. 2, template 20 has a marking for a first pre-measured distance 18 at 7 cm, a marking for a second pre-measured distance 28 at 10 cm and a marking for a third pre-measured distance 30 at 14 cm. These distances are only exemplary and one skilled in the art will recognize that additional distances may be marked as needed for proper neuromuscular evaluation, such as, but not limited to 5 cm, 6 cm and 12 cm.

The method for determining the stimulation point of peripheral nerves depicted in FIG. 2 has many advantages over the conventional method depicted in FIG. 1. The method depicted in FIG. 2 is more efficient and accurate than the conventional method depicted in FIG. 1. For example, a user does not need to manually measure a first distance 18 with a ruler 2, as shown in FIG. 1. Using the invention, a user simply places the ground electrode 14, reference electrode 12 and active electrode 10. Template 20, attached to the active electrode 10, directs the user where to place the simulation probes of waveform generator 16, as shown in FIG. 2. The user places the anode terminal in pre-punched holes 24 and the cathode terminal in pre-punched holes 26 to begin evaluation at the proper first pre-measured distance 18. As such, less time is wasted and human error is minimized by eliminating the need for manually measuring a first pre-measured distance 18. As a result, the method depicted in FIG. 2 is more efficient and accurate.

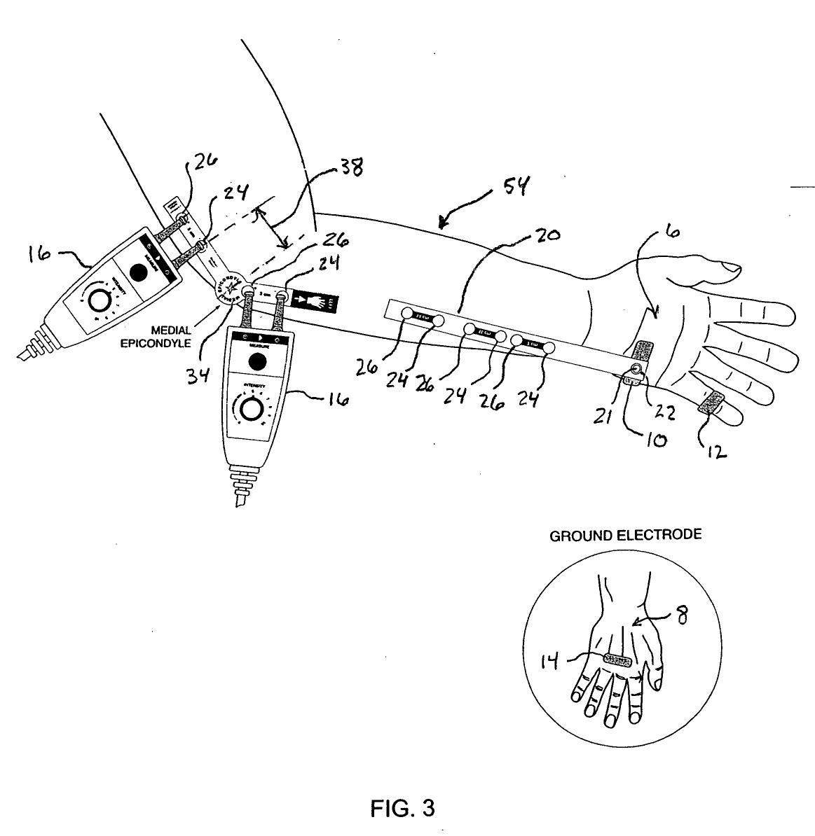

Alternative embodiments for using variations of template 20 are shown in FIG. 3. FIG. 3 depicts a V-shaped template 32 for quickly and accurately determining the stimulation point of an ulnar nerve across the elbow of a subject's left arm 54. Template 32 uses a the medial epicondyle as a reference point 34 for placement. The fourth pre-measured distances 38 is measured from the center of reference point 34. Again, template 32 has a plurality of pre-punched holes 24, 26 for receiving the stimulation probes of waveform generator 16. The waveform generator 16 can be, for example, a nerve conduction monitor or NCV/EMG device. The plurality of pre-punched holes 24 receives the anode terminal of the waveform generator 16 and the plurality of pre-punched holes 26 receives the cathode terminal of the waveform generator 16. The distance between the stimulation probe's anode and cathode on waveform generator 16 will correspond to the distance between pre-punched holes 24 and pre-punched holes 26. For example, if the distance from the center point of the stimulator's anode to the center point of the cathode is 2 cm, it will align with pre-punched holes 24 and pre-punched holes 26, respectively, that are also 2 cm on center. A user simply places the anode and cathode terminals of waveform generator 16 in pre-punched holes 24 and 26, respectively, to begin neuromuscular evaluation.

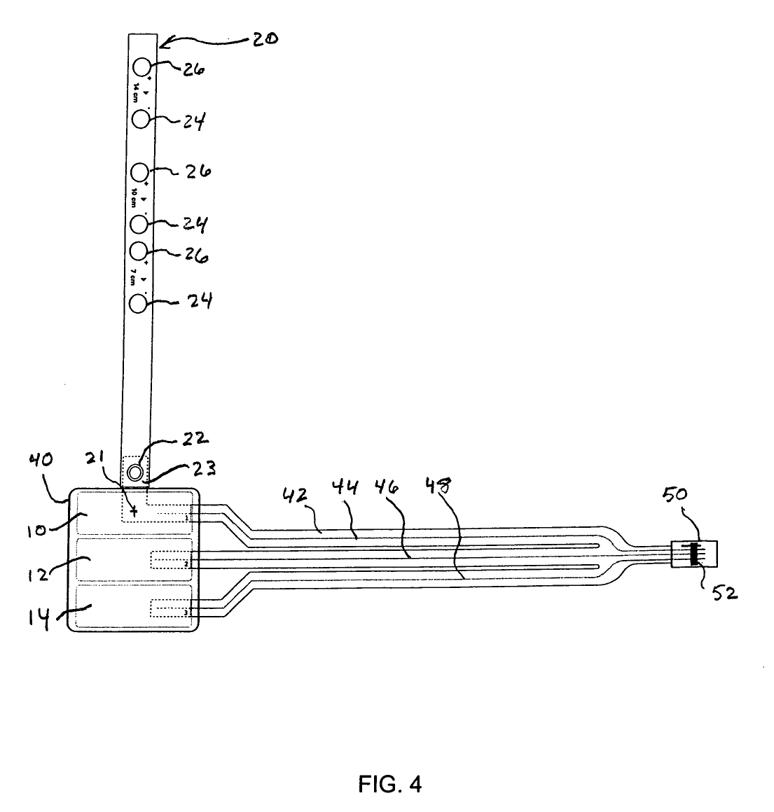

An illustration of the apparatus used in the methods described in FIGS. 2 and 3 is shown in more detail in FIG. 4. A plan view of the apparatus shows a substantially straight template 20 with a plurality of pre-punched holes 24, 26 for receiving the stimulation probes of a waveform generator (not shown). The plurality of pre-punched holes 24, 26 are formed in the template 20 along a radial axis perpendicular to the axis of rotation of fastener 22.

Each of the plurality of pre-punched holes 24, 26 are marked to receive either the simulator probe's anode or cathode. For example, the plurality of pre-punched holes 24 receives the anode terminal of the waveform generator 16 and the plurality of pre-punched holes 26 receives the cathode terminal of the waveform generator 16. The distance between the stimulation probe's anode and cathode on waveform generator (not shown) will correspond to the distance between hole 24 and hole 26. For example, if the distance from the center point of the stimulator's anode to the center point of the cathode is 2 cm, it will align with a hole 24 and hole 26, respectively, that are also 2 cm on center.

Moreover, template 20 has pre-measured distances are marked along template 20, for example a first pre-measured distance 18 at 7 cm, a second pre-measured distance 28 at 10 cm and a third pre-measured distance 30 at 14 cm. The distances are measured from the reference point 21, which may optionally correspond to the attachment or pivot point of fastener 22, to the center point of a corresponding pre-punched hole for the cathode (negative) probe. These distances are only exemplary and one skilled in the art will recognize that additional distances may be marked as needed for proper neuromuscular evaluation, such as, but not limited to 5 cm, 6 cm and 12 cm.

In one embodiment, template 20 is attached to the active electrode 10 by a fastener 22. The fastener 22 allows template 20 to rotate freely 360 degrees for accurate placement. The fastener 22 can be any axial fastener and may be aligned to the center of electrode 10 or offset from the electrode such as on an offset tab 23. Active electrode 10, reference electrode 12 and ground electrode 14 are placed on a release liner 40 until the electrodes are used.

Active electrode 10, reference electrode 12 and ground electrode 14 are connected to a circular connector 50 by circuits 44, 46 and 48, respectively. Alternatively, circuits 44, 46 and 4S can be a flattened wire, electrical trace, printed circuit or any other means to carry electrical signals. In the illustrated embodiment, the circuits 44, 46 and 48 are encapsulated on a flexible unitary substrate material 42, such as mylar. Use of the unitary substrate permits efficient manufacture of the apparatus. The circular connector 50 is used to connect the apparatus to a waveform generator (not shown).

Connector 50 contains a mechanical use limiter 52. The mechanical use limiter 52 may be, for example, a SMART LIMITER manufactured by MEDCONX of Santa Ana, Calif. Mechanical use limiter 52 ensures that the apparatus is not re-used beyond a pre-determined number of times. Re-use of the apparatus on multiple subjects may lead to reduced accuracy of the neuromuscular evaluation because of 1) the electrochemical gel being exposed to the open air for an extended or indefinite period of time; 2) increased electrical impedance from foreign particles, such as skin cells and natural oils released by a subject, adhering to the electrochemical gel; and physical distortion caused by adhering and removing the electrodes from subject to subject.

The mechanical use limiter 52 in circular connector 50 is comprised of many parts. An input path for electrical signals using circuits 44, 46, and 48 that travels through a positionable conductive member to an output path for electrical signals sent to a waveform generator (not shown). In addition, the mechanical use limiter 52 contains an adjustable potentiometer. A mechanical trigger, comprised of a spring assembly, in the mechanical use limiter 52 can be actuated to position the conductive member or adjust the potentiometer to control electrical communications between the output path and the input path.

The mechanical use limiter 52 is unidirectional and cannot be physically or electronically reversed. The mechanical use limiter 52 in the circular connector 50 allows electrical communication between the circuits 44, 46 and 48 and the waveform generator (not shown). Each time the connector 50 is connected securing the circuits 44, 46 and 48 to the waveform generator (not shown), or the connector 50 is disconnected removing the circuits 44, 46 and 48 from the waveform generator (not shown), constitutes an “occurrence”. At each occurrence, as defined herein to mean either connection or disconnection, the mechanical use limiter 52 changes to indicate ‘used’ or increments a counter higher. The mechanical use limiter 52 allows communication between the circuits 44, 46 and 48 and the waveform generator (not shown) until a predetermined number of occurrences is reached. Once the number of occurrences is reached, the mechanical trigger in mechanical use limiter 52 is actuated. Once actuated, the mechanical use limiter 52 connects two circuits using an electrical component, such as a resistor, thereby causing the active and reference circuits to short out, for example. An electrical response captured after the predetermined number of occurrences is reached will have a specific waveform configuration that, when recognized by the waveform generator, indicates the apparatus can no longer be used. The apparatus can never be used again unless the mechanical use limiter 52 is physically replaced. Therefore, the apparatus is disposed of and a new apparatus is required for further neuromuscular evaluation. As a result, the mechanical use limiter 52 is more secure for ensuring limited re-use of the apparatus compared to other available technologies.

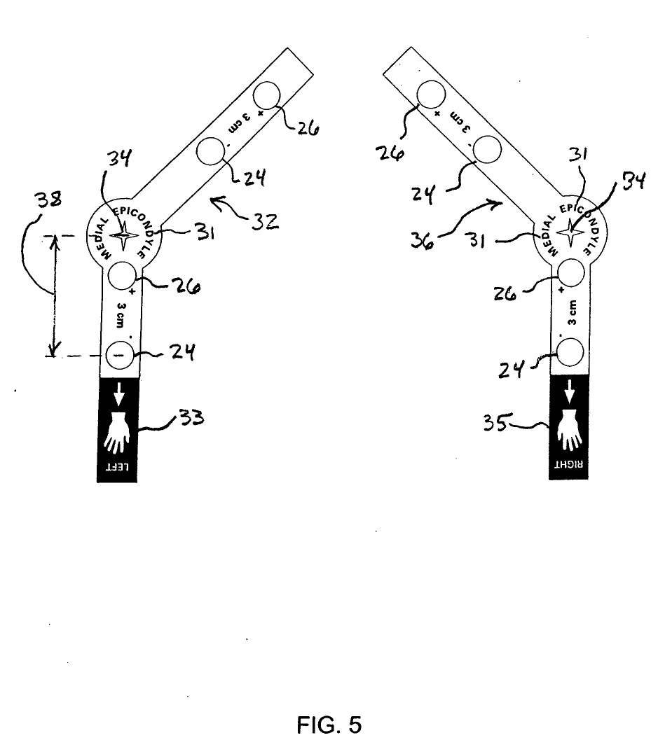

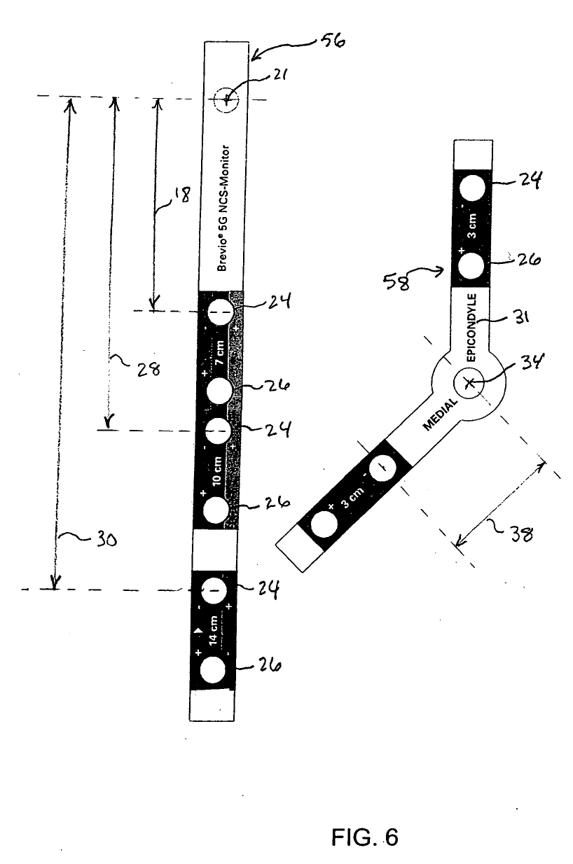

An illustration of various design embodiments for templates used to determine the stimulation point of various nerves are shown on FIGS. 5 and 6. FIG. 5 illustrates a V-shaped template 32 for determining the stimulation point of the ulnar nerve across the left elbow and a V-shaped template 36 for the right elbow. Left and right templates 32 and 36, respectively, use the medial epicondyle as a reference point 34 to fourth pre-measured distances 38. Again, right and left templates 32 and 36, respectively, have a plurality of pre-punched holes 24, 26 for receiving the stimulation probes of a waveform generator (not shown). The plurality of pre-punched holes 24 and 26 guide the user for quick and efficient placement of a simulation probe's anode and cathode to obtain accurate results. The templates shown in FIG. 5 include a reference point indicia 31 indicating the intended body part location for placement of reference point 34. In these exemplary embodiments, the reference point is indicated to be the medial epicondyle. Other templates would similarly indicate the intended placement of the reference point. Also, the proper orientation of the template is preferably indicated by orientation indicia 33 and 35, respectively, on the template.

FIG. 6 illustrates an ambidextrous template 56 for peripheral nerves and template 58 for ulnar nerves. Template 56 can be used for either the left or right arm. Template 58 can be used for either the left or right elbow. Template 56 is similar to template 20, shown in FIG. 4, in all respects. Additionally, template 56 has a positive symbol “+” for the anode and negative symbol “−” for the cathode on both edges to indicate the correct placement of the stimulation probes. Whether the plurality of pre-punched holes 24 and 26 receive the stimulation probe's anode or cathode depends on which arm template 56 is used on. Although embodiments of the template have been described for the arm, one skilled in the art could modify the template for evaluation of neuromuscular function in the leg or other parts of the body.

Furthermore, various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed is:

1. Apparatus for sensing a neuromuscular function signal, comprising:
a plurality of electrodes including an active electrode;
a plurality of electrical circuits, each on of the plurality of electrical circuits in electrical communication with one of the plurality of electrodes;
a connector for securing the plurality of electrical circuits to a waveform generator interface; and
a template having a reference point positionable relative to the active electrode and a visual indicator along the template at a predetermined distance from the reference point indicating the desired positions of a stimulation probe.

2. The apparatus of claim 1, wherein the reference point corresponds to an attachment point at which the template is attached to the active electrode.

3. The apparatus of claim 1, further comprising a fastening device attaching the template to the active electrode.

4. The apparatus of claim 3, wherein the fastening device is positioned on an offset tab attached to the active electrode.

5. The apparatus of claim 1, wherein the visual indicator comprises a pair of holes formed along the template.

6. The apparatus of claim 5, wherein the pair of holes are sized for passage of the pair of stimulator probes there through.

7. The apparatus of claim 1, wherein the template comprises a set of characters representative of the predetermined distance.

8. The apparatus of claim 1, wherein the plurality of electrical circuits comprise at least one of a plurality of substrate-encapsulated electrical traces, a plurality of substrate-encapsulated flattened wires, and a plurality of flexible wires.

9. The apparatus of claim 1, wherein the template has polarity indicia disposed thereon and indicative of the stimulator probe polarity corresponding to the intended placement of a pair of stimulator probes.

10. The apparatus of claim 1, wherein the apparatus further comprises a connector having a mechanical use limiter allowing electrical communication between the plurality of electrical circuits and the waveform generator interface during each instance of the connector securing the plurality of electrical circuits to the waveform generator interface until a predetermined number of said instances have occurred.

11. The apparatus of claim 10, wherein the mechanical use limiter comprises:
an output path for electrical communication with the waveform generator interface;
an input path for electrical communication with the plurality of electrical circuits; and
a conductive member positionable to allow electrical communication between the output path and the input path; and
a mechanical trigger in mechanical communication with the conductive member, the mechanical trigger being actuated upon each instance of the connector securing the plurality of electrical circuits to the waveform generator interface, and the mechanical trigger, in response to being actuated the predetermined number of instances, positioning the conductive portion to allow electrical communication between the output path and the input path.

12. The apparatus of claim 11, wherein the mechanical trigger comprises a spring assembly.

13. The apparatus of claim 11, wherein the mechanical use limiter comprises:
an output path for electrical communication with the waveform generator interface;
an input path for electrical communication with the plurality of electrical circuits; and
a potentiometer adjustable to allow electrical communication between the output path and the input path; and
a mechanical trigger in mechanical communication with the potentiometer and actuated upon each instance of the connector securing the plurality of electrical circuits to the waveform generator interface, the mechanical trigger, in response to being actuated the predetermined number of instances, adjusting the potentiometer to allow electrical communication between the output path and the input path.

14. The apparatus of claim 1, comprising an electronic use limiter.

15. The apparatus of claim 14, wherein the electronic use limiter comprises a data memory for storing a signal representative of an indication that the apparatus has been used to acquire a neuromuscular function signal.

16. Apparatus for sensing a neuromuscular function signal, comprising:
a plurality of electrodes including an active electrode;
a plurality of electrical circuits, each one of the plurality of electrical circuits in electrical communication with one of the plurality of electrodes;
a connector for securing the plurality of electrical circuits to a waveform generator interface; and
a template attached to the active electrode, a pair of holes being formed along the template at a predetermined distance from a reference point and being sized for passage of a pair of stimulation probes there through; and
a fastening device attaching the template to the active electrode.

17. The apparatus of claim 16, wherein the fastening device comprises an axial fastener attaching the template to the active electrode.

18. The apparatus of claim 16, wherein the fastening device comprises:
an offset tab attached to the active electrode; and
an axial fastener attaching the template at the pivot point to the offset tab.

19. The apparatus of claim 16, wherein the plurality of electrodes further includes a reference electrode and a ground electrode.

20. The apparatus of claim 16, wherein the plurality of electrical circuits comprise at least one of a plurality of substrate-encapsulated electrical traces and a plurality of substrate-encapsulated flattened wires.

21. The apparatus of claim 16, wherein the template has a second pair of holes formed at a second predetermined distance from the reference point and sized for passage of the pair of stimulator probes there through, and wherein the template has a second set of characters representative of the second predetermined distance and disposed thereon proximate to the second pair of holes.

22. The apparatus of claim 21, wherein the first pair of holes is formed in the template along a radial axis, and wherein the second pair of holes is formed in the template along the radial axis.

23. The apparatus of claim 22, wherein the template is substantially straight.

24. The apparatus of claim 21, wherein the first pair of holes is formed in the template along a radial axis perpendicular to the axis of rotation, and wherein the second pair of holes is formed in the template along another radial axis perpendicular to the axis of rotation.

25. The apparatus of claim 24, wherein the template is substantially V-shaped.

26. The apparatus of claim 16, wherein the predetermined distance comprises a member of the group consisting of three centimeters, five centimeters, six centimeters, seven centimeters, ten centimeters, twelve centimeters, and fourteen centimeters.

27. The apparatus of claim 16, wherein the center points of each hole in the pair of holes are spaced about two centimeters from one another.

28. The apparatus of claim 16, wherein the template has polarity indicia disposed thereon and indicative of the stimulation probe polarity corresponding to each hole in the pair of holes.

29. The apparatus of claim 16, wherein the connector comprises a mechanical use limiter allowing electrical communication between the plurality of electrical circuits and the waveform generator interface during each instance of the connector securing the plurality of electrical circuits to the waveform generator interface until a predetermined number of said instances have occurred.

30. The apparatus of claim 29, wherein the mechanical use limiter comprises:
an output path for electrical communication with the waveform generator interface;
an input path for electrical communication with the plurality of electrical circuits; and
a conductive member positionable to allow electrical communication between the output path and the input path; and
a mechanical trigger in mechanical communication with the conductive member, the mechanical trigger being actuated upon each instance of the connector securing the plurality of electrical circuits to the waveform generator interface, and the mechanical trigger, in response to being actuated the predetermined number of instances, positioning the conductive portion to allow electrical communication between the output path and the input path.

31. The apparatus of claim 30, wherein the mechanical trigger comprises a spring assembly.

32. The apparatus of claim 30, wherein the mechanical use limiter comprises:
an output path for electrical communication with the waveform generator interface;
an input path for electrical communication with the plurality of electrical circuits; and
a potentiometer adjustable to allow electrical communication between the output path and the input path; and
a mechanical trigger in mechanical communication with the potentiometer and actuated upon each instance of the connector securing the plurality of electrical circuits to the waveform generator interface, the mechanical trigger, in response to being actuated the predetermined number of instances, adjusting the potentiometer to allow electrical communication between the output path and the input path.

33. The apparatus of claim 16, comprising an electronic use limiter.

34. The apparatus of claim 33, wherein the electronic use limiter comprises a data memory for storing a signal representative of an indication that the apparatus has been used to acquire a neuromuscular function signal.

35. Apparatus for acquiring a neuromuscular function signal, comprising:
a plurality of electrodes including an active electrode;
a plurality of electrical circuits, each one of the plurality of electrical circuits in electrical communication with one of the plurality of electrodes;
a connector for securing the plurality of electrical circuits to a waveform generator interface, wherein the connector comprises a mechanical use limiter allowing electrical communication between the plurality of electrical circuits and the waveform generator interface during each instance of the connector securing the plurality of electrical circuits to the waveform generator interface until a predetermined number of said instances have occurred.

36. The apparatus of claim 35, wherein the mechanical use limiter comprises:
an output path for electrical communication with the waveform generator interface;
an input path for electrical communication with the plurality of electrical circuits; and
a conductive member positionable to allow electrical communication between the output path and the input path; and
a mechanical trigger in mechanical communication with the conductive member, the mechanical trigger being actuated upon each instance of the connector securing the plurality of electrical circuits to the waveform generator interface, and the mechanical trigger, in response to being actuated the predetermined number of instances, positioning the conductive portion to allow electrical communication between the output path and the input path.

37. The apparatus of claim 35, wherein the mechanical use limiter comprises:
an output path for electrical communication with the waveform generator interface;
an input path for electrical communication with the plurality of electrical circuits; and
a potentiometer adjustable to allow electrical communication between the output path and the input path; and
a mechanical trigger in mechanical communication with the potentiometer and actuated upon each instance of the connector securing the plurality of electrical circuits to the waveform generator interface, the mechanical trigger, in response to being actuated the predetermined number of instances, adjusting the potentiometer to allow electrical communication between the output path and the input path.

38. Apparatus for acquiring a neuromuscular function signal, comprising:
a plurality of electrodes including an active electrode, a reference electrode, and a ground electrode;
a plurality of electrical circuits, each one of the plurality of electrical circuits in electrical communication with one of the plurality of electrodes;
a connector for securing the plurality of electrical circuits to a waveform generator interface; and
a template having a reference point positionable relative to the active electrode and a visual indicator along the template at a predetermined distance from the reference point indicating the desired positions of a pair of stimulation probes.

39. The apparatus of claim 38, wherein the connector comprises a mechanical use limiter allowing electrical communication between the plurality of electrical circuits and the waveform generator interface during each instance of the connector securing the plurality of electrical circuits to the waveform generator interface until a predetermined number of said instances have occurred.

40. The apparatus of claim 38, comprising an electronic use limiter having a data memory.

41. A template for positioning a stimulation probe for sensing a neuromuscular function signal, comprising:
a strip having a reference point; and
a visual indicator along the strip a predetermined distance from the reference point and indicating the desired placement of the stimulation probe.

42. The template of claim 41, wherein the reference point is positionable relative to an active electrode of an apparatus for sensing a neuromuscular function signal.

43. The template of claim 41, wherein the reference point is positionable relative to a predetermined body part.

44. The template of claim 43, wherein the body part is the medial epicondyle.

45. The template of claim 41, wherein the template comprises a pair of visual indicators for indicating the desired placement of a pair of stimulator probes

46. The template of claim 45, wherein the visual indicator comprises a pair of holes formed along the template.

47. The template of claim 46, wherein the pair of holes are sized for passage of a pair of stimulation probes there through.

48. The template of claim 41, wherein the template comprises a set of characters representative of the predetermined distance.

49. The template of claim 41, wherein the template has polarity indicia disposed thereon and indicative of the stimulation probe polarity.

50. The template of claim 41, wherein the template has orientation indicia indicative of the proper orientation of the template.

51. The template of claim 41, wherein the template has reference point indicia indicative of the intended placement of the reference point on a human body.
